(19) United States
(12) Patent Application Publication
(10) Pub. No.: US 2021/0222310 A1
(43) Pub. Date: Jul. 22, 2021

(54) PREPARATION METHOD OF MINIATURE INTELLIGENT CALCIUM ALGINATE HYDROGEL END OPERATOR

(71) Applicant: BEIJING INSTITUTE OF TECHNOLOGY, Beijing (CN)

(72) Inventors: HUAPING WANG, Beijing (CN); QING SHI, Beijing (CN); SHAOAN WANG, Beijing (CN); ZHIQIANG ZHENG, Beijing (CN); QIANG HUANG, Beijing (CN); TOSHIO FUKUDA, Beijing (CN)

(21) Appl. No.: 17/142,264

(22) Filed: Jan. 6, 2021

Related U.S. Application Data
(63) Continuation-in-part of application No. PCT/CN2020/073728, filed on Jan. 22, 2020.

(51) Int. Cl. C25D 1/20; A61M 31/00; C09D 5/44; C25D 1/08; C25D 5/00; C25D 5/34; C08L 5/04

(52) U.S. Cl. CPC C25D 1/20; A61M 31/002; C09D 5/44; C25D 1/08; A61M 2205/02; C25D 5/34; C25D 5/605; C08L 5/04; A61M 2207/00; C25D 5/007

(57) ABSTRACT

A preparation method of a miniature intelligent calcium alginate hydrogel end operator based on different microelectrodes is introduced. The method includes an electrodeposition step of depositing a deposition solution under the action of a non-uniform magnetic field to form an anode surface; a processing step of transferring obtained hydrogel microstructures to a calcium chloride solution, making the hydrogel microstructure self-wind sufficiently; and a pickup step of collecting a self-winding single-layer film alginate microstructure in a culture dish, and placing it in specific environment for preservation. The preparation method can provide a degradable and convenient micro-operator, which could be locally prepared into different function components.

### CROSS-REFERENCE TO RELATED APPLICATION

This application is a continuation-in-part patent application of PCT application No. PCT/CN2020/073728 filed on Jan. 22, 2020, the entire contents of which are hereby incorporated by reference for which priority is claimed under 35 U.S.C. § 365

### BACKGROUND OF THE INVENTION

### 1. Field of the Invention

The present disclosure relates to the technical field of drug microcarriers, particularly to a preparation method of a miniature intelligent calcium alginate hydrogel end operator based on different microelectrodes.

### 2. Description of the Related Art

Drug delivery refers to delivery of drugs to lesions in a specific way, so as to achieve the purpose of treating diseases. Common clinical drug delivery manners include enteral administration, parenteral administration, and local administration, etc. Oral administration and rectal administration are two manners of enteral administration, in which drugs enter the blood after being absorbed by gastrointestinal mucosa, liver or rectal mucosa, and then transported to the whole body through blood circulation, so as to achieve the purpose of treating diseases. Intravenous injection, intramuscular injection and subcutaneous injection are three common manners of parenteral administration, in which drugs are directly injected into veins, muscle tissue and subcutaneous tissue. The local administration manner means to deliver drugs through eyes, lungs, abdominal cavity and skin to the lesion directly, so as to achieve the purpose of treating diseases. Compared with systemic administration, the local administration can obtain higher drug concentration and fewer side effects, thereby bringing about significant advantages. Although the traditional drug delivery can play a certain role in the diagnosis and treatment of some specific diseases, most of them are faced with the problems of rapid drug release, short drug action time, and long-term and multiple drug administration, etc. In order to solve these problems, in recent years, with the rapid development of medicine, chemistry, materials, engineering, information, life and other sciences, drug microcarrier preparation technology has attracted more and more attention from researchers domestically and abroad. Combining drugs with microcarriers to form a microcarrier drug delivery system has the characteristics of targeted controlled release, safety, reliability and modifiability. Appropriate types of drug microcarriers selected upon clinical requirements can not only provide fixed-point deliver drugs to target organs, but also effectively adjust physicochemical properties of the drugs, thereby achieving the effects of improving therapeutic effects, reducing toxicity and side effects, reducing treatment cost and so on. At present, the research of drug microcarriers mainly focuses on such aspects as microspheres, microcapsules, nanoparticles, liposomes, microemulsions, microvesicles, and inclusion compounds, etc.

However, the existing drug carrier preparation technology has many technical barriers, such as complex devices, difficult preparation and non-degradability. Therefore, in the field of precise medicine and tissue engineering, there is need of a degradable and convenient micro-operator, which can be locally prepared into different function components.

### BRIEF SUMMARY OF THE INVENTION

A purpose of the present invention is to provide a preparation method of a miniature intelligent calcium alginate hydrogel end operator based on different microelectrodes.

A technical solution of the present invention is as follows:

The present invention provides a micro-operator for processing different structures based on different microelectrodes, so as to realize a preparation method of a miniature intelligent calcium alginate hydrogel end operator with different functions, including: electrodeposition, processing and pickup.

Preferably, the electrodeposition further includes:

a1: coating a photoresist on an FTO glass by a spin coater, and forming different microelectrodes by photolithography;

a2: filling a deposition solution between two electrodes and maintaining the deposition solution by two insulating spacers with a height of 1 mm;

a3: applying direct voltage to the electrodes for 1 to 5 seconds; and

a4: washing an anode in an HEPES buffer solution for 5 minutes until all hydrogel microstructures are separated from the ITO glass.

Preferably, the electrodeposition step further includes:

a1: coating a photoresist on an FTO glass by a spin coater, and forming a concave pattern with a specific shape on the FTO glass as an anode;

a2: filling a deposition solution between two electrodes and maintaining the deposition solution by insulating spacers;

a3: applying direct voltage to the electrodes for 1 to 5 seconds; and

a4: washing the anode in an HEPES buffer solution.

Preferably, in step a2, the number of the insulating spacers is not less than 2, and thickness of the insulating spacers is not less than 1 mm. Preferably, in step a4, the washing time is 3 to 10 minutes.

Preferably, the deposition solution includes CaCO₃, drugs, cells, magnetic nanoparticles and monitoring equipment.

Preferably, the processing further includes:

b1: transferring the hydrogel microstructures into a calcium chloride solution for 2 minutes, making the hydrogel microstructures self-wind sufficiently.

Preferably, the pickup further includes:

c1: collecting the microstructures in a culture dish; and

c2: placing the culture dish in a specific environment for preservation.

Preferably, the degradable and self-winding miniature intelligent calcium alginate hydrogel end operator is produced by the preparation method of a degradable and self-winding miniature intelligent calcium alginate hydrogel end operator.

In another aspect, the present invention provides an application of the miniature intelligent calcium alginate hydrogel end operator in the medical treatment.

The present invention provides a passive transportation application of the miniature intelligent calcium alginate hydrogel end operator comprising: the miniature intelligent calcium alginate hydrogel end operator that can enter into a digestive system of a human body through swallowing after gripping magnetic microspheres in vitro, and reach the lesion under manipulation of the magnetic field generated by the external magnetic control system. After the step of separating the magnetic microsphere from the miniature intelligent calcium alginate hydrogel end operator is completed at the lesion, the magnetic microspheres continue to move away from the human body through the intestinal tract under manipulation of the external magnetic field. The miniature intelligent calcium alginate hydrogel end operator stays at the lesion to complete the degradation process, and release the carried therapeutic drugs or liver cells with a repair function, etc.

The present invention also provides a positive transportation application of the miniature intelligent calcium alginate hydrogel end operator comprising: the miniature intelligent calcium alginate hydrogel end operator carrying with magnetic nanoparticles inside, with deformability and global motion performance, that can directly enter into the digestive system of the human body through swallowing. The miniature intelligent calcium alginate hydrogel end operator reaches the target area under drive of the external magnetic field, and completes gripping of a specific target in a target area. After completion of the gripping task, the miniature intelligent calcium alginate hydrogel end operator can transport the target as held in vitro along the digestive tract or a destination in another digestive tract.

Through the above technical solutions, the present invention produces a specific hydrogel through the electrodeposition module, and then forms a self-winding single-layer film alginate microstructure by processing the hydrogel. The microstructure can be located by a magnetic field and degraded in a sodium citrate solution. The present invention can provide a degradable and convenient micro-operator, which can be locally prepared into different function components.

### BRIEF DESCRIPTION OF THE DRAWINGS

FIGS. 1a and 1b are schematic diagrams of a micro-tissue construction device based on electrodeposition of the present invention; FIG. 1a is a schematic diagram of stripped hydrogel electrodeposition; and FIG. 1b is a schematic diagram of radial hydrogel electrodeposition.

FIGS. 2a and 2b are schematic diagrams of a single-layer film alginate microstructure; and FIG. 2a and FIG. 2b are stripped and radial hydrogel microstructures after electrodeposition, respectively.

FIGS. 3a to 3c are schematic diagrams of self-winding deformation of an alginate single layer film microstructure; FIG. 3a and FIG. 3b are schematic diagrams of shapes of the self-winding stripped hydrogel microstructure, respectively; and FIG. 3c is a schematic diagram of a shape of the self-winding radial hydrogel microstructure.

FIGS. 4a and 4b are schematic diagrams of a microelectrode to generate electric fields; FIG. 4a is a structure schematic diagram of a microelectrode to generate non-uniform electric fields; and FIG. 4b is a structure schematic diagram of a microelectrode to generate uniform electric fields.

### DETAILED DESCRIPTION OF THE INVENTION

In order to make those skilled in the art better understand the solution of the present application, the technical solutions in embodiments of the present application will be described clearly and comprehensively below with reference to accompanying drawings of the embodiments of the present application. Obviously, the described embodiments are merely some, rather than all, of the embodiments of the present application. Based on the embodiments of the present application, all other embodiments obtained by those ordinarily skilled in the art without making creative efforts should fall within the scope of protection of the present application.

The alginate hydrogel microstructures prepared based on the non-uniform electrodeposition technology can undergo reciprocal self-deformation under the action of ions or pH. Compared with the traditional uniform electrodeposition, the non-uniform electrodeposition does not only generate non-uniformity in the hydrogel space network in the thickness direction (Z direction), but also generate non-uniform space network on the X-Y plane in each layer. This non-uniform hydrogel space network exhibits a non-uniform expansion rate in the process of expansion or contraction. Therefore, through the design on the processed electric field in the non-uniform electrodeposition technology, the controllability on the non-uniform hydrogel space network is realized, and the alginate hydrogel microstructures are endowed with different motion abilities and motion modes, e.g., gripping, releasing, self-winding, etc.

In the present invention, a preparation method of the degradable and self-winding miniature intelligent calcium alginate hydrogel end operator generates a micro-robot end operator that generates deformation of the shape induced by ions using single degradable biomaterials for the first time, and a multi-field control robot system performs different motion modes and automatic transfer under various physical and chemical environments.

The present invention provides a preparation method of a degradable and self-winding miniature intelligent calcium alginate hydrogel end operator, including: electrodeposition, processing and pickup.

Preferably, the electrodeposition further includes:

In one preferable embodiment, the step S11 further includes:

S1: coating a photoresist with a thickness about 10 μm on an FTO glass with a size of 50 mm×50 mm by a spin coater, and forming a concave pattern with a specific shape on the FTO glass as an anode;

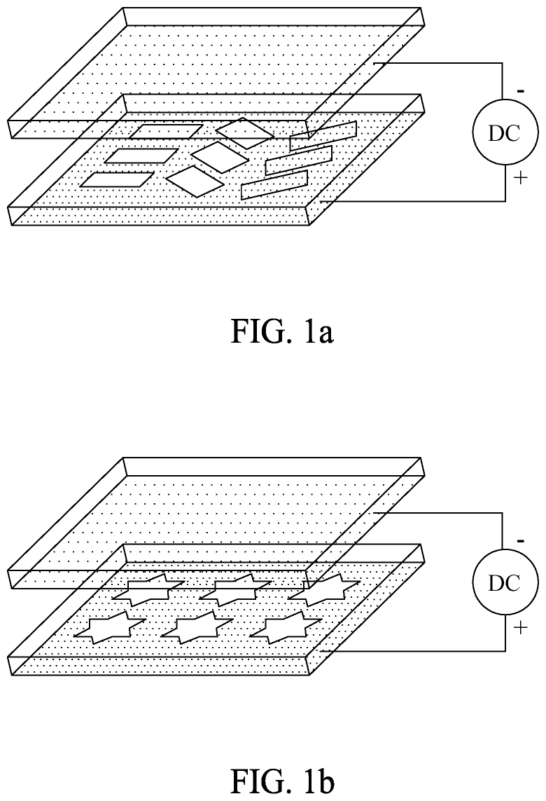

S2: filling 2 ml of a deposition solution between two electrodes of 3.5V to 4V (as shown in FIG. 1a and FIG. 1b) and maintaining the deposition solution by two insulating spacers with a height of 1 mm;

S3: applying direct voltage to the electrodes for 1 to 5 seconds; and

S4: washing the anode in an HEPES buffer solution for 5 minutes until all hydrogel microstructures are separated from the FTO glass.

In one preferable embodiment, the processing further includes:

S1: transferring the hydrogel microstructures to 0.5% calcium chloride solution for 2 minutes, making the hydrogel microstructures self-wind sufficiently, wherein the hydrogel microstructures can restore its shape and dissolve in a sodium citrate solution.

In one preferable embodiment, the pickup further includes:

S1: collecting the microstructures in a culture dish; and

S2: placing the culture dish in a specific environment for preservation.

In one preferable embodiment, the required component includes CaCO₃, and the method includes drugs, cells, magnetic nanoparticles and monitoring equipment.

1. Deformation speed of the degradable and self-winding miniature intelligent calcium alginate hydrogel end operator of the present invention with different pHs, different CaCl₂) and sodium citrate densities:

Experiment method: the degradable and self-winding miniature intelligent calcium alginate hydrogel end operator is sucked out with a dropper and is dripped in the corresponding test solution quickly (the test solutions are: 90 mmol/L CaCl₂ solution, 900 mmol/L CaCl₂ solution, 90 mmol/L sodium citrate solution and 900 mmol/L sodium citrate solution; solution with pH 1 to 3, solution with pH 5 to 11 and solution with pH 4).

Notes: the CaCl₂ solution and the sodium citrate solution are prepared by corresponding solid powder and deionized water; different pH solutions are prepared by sodium hydroxide and concentrated hydrochloric acid and determined by the pH meter.

It can be obtained by the experiment that: as for the degradable and self-winding miniature intelligent calcium alginate hydrogel end operator of the present invention, the calcium alginate hydrogel end operator needs 32 s to complete contraction and deformation when the density of CaCl₂ is 90 mmol/L, and reduces the deformation time to 11 s when the density of CaCl₂ increases to 900 mmol/L, wherein the same tendency is also observed for the release motion.

When densities of the sodium citrate solution are 90 mmol/L and 900 mmol/L, the calcium alginate hydrogel end operator uses 13 s and 2.5 s to complete the diastolic expansion process, respectively. The permeation range of cells in vivo is 290 to 310 mmol/L, which provides a sufficient range of the ionic strength, and controls the shape change and dissolution by the ion change;

The alginate hydrogel microstructure contracts when pH is 1 to 3, expands when pH is 5 to 11 and has no change when pH is about 4.

2. Influence on deformation of the degradable and self-winding miniature intelligent calcium alginate hydrogel end operator of the present invention by the current density range:

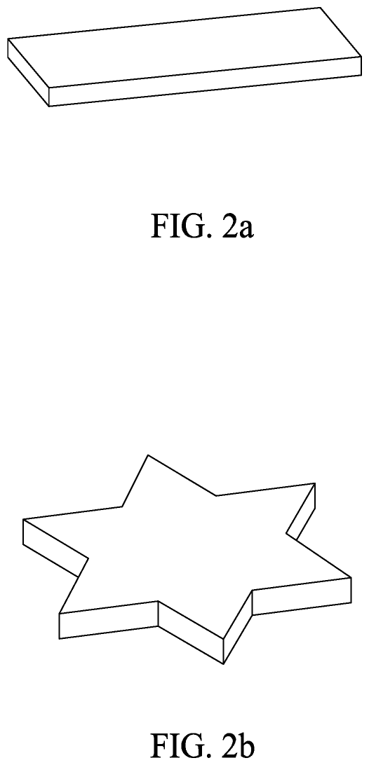

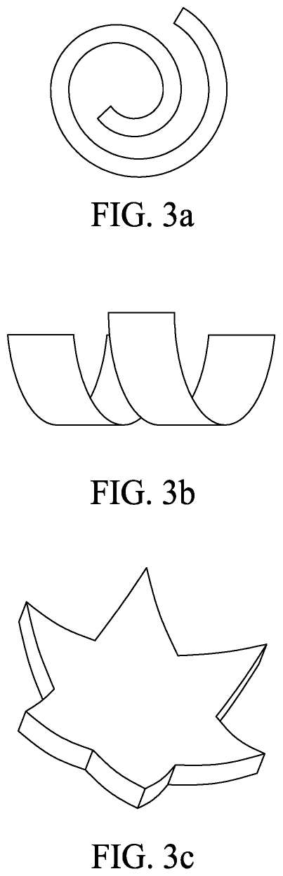

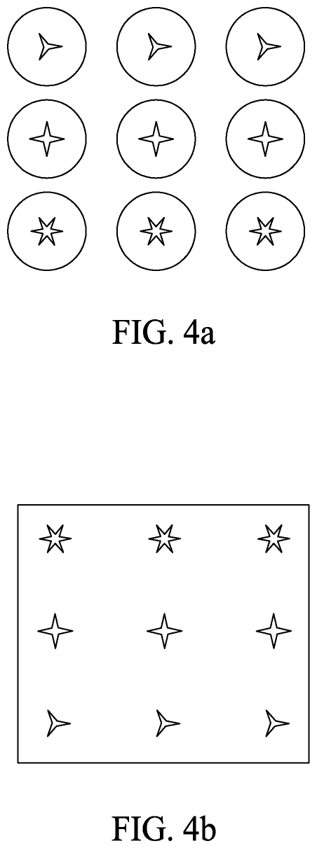

Experiment method: 2 ml of deposition solution mixed with fluorescent nanoparticles is dripped on a positive plate between two electrodes. Secondly, 3-4V of direct voltage is applied on two layers of FTO electrodes, for a duration time of 1 to 10 s. After completion of the electrodeposition, the HEPES buffer solution is used to wash for more than 3 min in 10 cm of the culture dish, until the calcium alginate hydrogel microstructure is completely separated from the electrode. The alginate hydrogel microstructure generated by different electrodes (microelectrode of the non-uniform electric field, as shown in FIG. 4a or microelectrode of the uniform electric field, as shown in FIG. 4b) is sucked out by the dropper, and the alginate microstructure is transferred to another 6 cm of the culture dish containing the HEPES buffer solution. The CaCl₂) solution (1.1% w/v) is slowly injected, until the shape completely changes (the changed shape is shown in FIGS. 2a, 2b, 3a, 3b, 3c).

According to the above experiment, it can be obtained that the calcium alginate hydrogel end operator can have the deformability only when the thickness is about 150 to 300 microns; and the processing conditions must be that the current density range is 3 to 4V, the deposition time is 2 to 9 s, otherwise the calcium alginate hydrogel end operator does not have the deformability under the microelectrode processing condition of the uniform electronic field.

The above-mentioned are only specific examples of the invention, which are only used to explain the technical solution and the inventive concept of the present invention, instead of limiting the scope of the claims of the invention. Any other technical solutions that can be obtained by those skilled in the art through logical analysis, reasoning or limited experiments on the basis of the inventive concept of the present patent in combination with the prior art shall also be deemed to fall within the protection scope of the claims of the present invention.

While the present disclosure has been described by means of specific embodiments, numerous modifications and variations could be made thereto by those skilled in the art without departing from the scope and spirit of the present disclosure set forth in the claims.

What is claimed is:

1. A preparation method of a degradable and self-winding miniature intelligent calcium alginate hydrogel end operator, comprising: electrodeposition, processing and pickup.

2. The preparation method of a degradable and self-winding miniature intelligent calcium alginate hydrogel end operator according to claim 1, characterized in that: the electrodeposition step further comprises:
a1: coating a photoresist on an FTO glass by a spin coater, and forming a concave pattern with a specific shape on the FTO glass as an anode;
a2: filling a deposition solution between two electrodes and maintaining the deposition solution by insulating spacers with a height of 1 mm;
a3: applying direct voltage to the electrodes for 1 to 5 seconds; and
a4: washing the anode in an HEPES buffer solution for 5 minutes until all hydrogel microstructures are separated from the FTO glass.

3. The preparation method of a degradable and self-winding miniature intelligent calcium alginate hydrogel end operator according to claim 1, characterized in that the electrodeposition step further comprises:
a1: coating the photoresist on the FTO glass by the spin coater, and forming the concave pattern with the specific shape on the FTO glass as the anode;
a2: filling the deposition solution between the two electrodes and maintaining the deposition solution by the insulating spacers;
a3: applying direct voltage to the electrodes for 1 to 5 seconds; and
a4: washing the anode in the HEPES buffer solution.

4. The preparation method of a degradable and self-winding miniature intelligent calcium alginate hydrogel end operator according to claim 3, characterized in that in step a2, the number of the insulating spacers is not less than 2, thickness of the insulating spacers is not less than 1 mm, and in step a4, the washing time is 3-10 minutes.

5. The preparation method of a degradable and self-winding miniature intelligent calcium alginate hydrogel end operator according to claim 3, characterized in that the deposition solution includes CaCO₃, drugs, cells, or magnetic nanoparticles.

6. The preparation method of a degradable and self-winding miniature intelligent calcium alginate hydrogel end operator according to claim 1, characterized in that the processing step further comprises:
b: transferring the hydrogel microstructures into the calcium chloride solution for 2 minutes, making the hydrogel microstructures self-wind sufficiently.

7. The preparation method of a degradable and self-winding miniature intelligent calcium alginate hydrogel end operator according to claim 1, characterized in that the pickup step further comprises:
c1: collecting the microstructures in a culture dish; and
c2: placing the culture dish in a specific environment for preservation.

8. An application of the miniature intelligent calcium alginate hydrogel end operator according to claim 3 in medical treatment.
